SEQUENCE LISTING (6 sequences)

SEQ ID NO: 1 - DNA, 3343 bp, Homo sapiens
gagctcctggccagtggtggagagtggcaaggaaggaccctagggttcatcggagcccaggtttactcccttaagtggaaatttcttcccccactcccctccttggctttctccaaggagggaaccccaggctgctggaaagtccggctggggcggggactgtgggtttcagggtagaactgcgtgtggaacgggacagggagcggttagaagggtggggctattccgggaagtggtggtggggggagggagcccaaaactagcacctagtccactcattatccagccctcttatttctcggccgcctctgcttcagtggacccggggagggcggggaagtggagtgggagacctaggggtgggcttcccgaccttgctgtacaggacctcgacctagctggctttgttccccatccccagttagttgttgccctgaggctaaaactagagcccaggggccccaagttccagactgcccctcccccctcccccggagccagggagtggttggtgaaagggggaggccagctggagaagaaacgggtagtcaggggttgcagcattagagcccttgtagccctagcccaggaatggttggagagagaagagtagagtagggaggggggtttgtcacctgtcacctgctcggctgtgcctagggcgggcgggggggagtggggggaccggtataaagcggtaggcgcctgtgcccgctccacctctcaagcagccagcgcctgcctgaatctgttctgccccctccccacccatttcaccaccaccatgacaccgggcacccagtctcctttcttcctgctgctgctcctcacagtgcttacaggtgaggggcacgaggtggggagtgggctgccctgcttaggtggtcttcgtggtctttctgtgggttttgctccctggcagatggcaccagaagttaaggtaagaattgcagacagaggctgccctgtctgtgccagaaggagggagaggctaaggacaggctgagaagagttgcccccaaccctgagagtgggtaccaggggcaagcaaatgtcctgtagagaagtctagggggaagagagtagggagagggaaggcttaagaggggaagaaatgcaggggccatgagccaaggcctatgggcagagagaaggaggctgctgcaggaaggaggcggccaacccaggggttactgaggctgcccactccccagtcctcctggtattatttctctggtggccaggcttatattttcttcttgctcttatttttccttcataaagacccaaccctatgactttaacttcttacagctaccacagcccctgggcccgcaacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccagggtgtcacctcggccccggagaccaggccgcccccgggctccaccgcccccccagcccatggtgtcacctcggcgccggacaacaggcccgccttggcgtccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatggtgagtatcggcctttccttccccatgctcccctgaagcagccatcagaactgtccacaccctttgcatcaagcctgagtcctttccctctcaccccagtttttgcagatttataaacaagggggttttctgggcctctccaatattaagttcaggtacagttctgggtgtggacccagtgtggtggttggaggggtgggtggtggtcatgagccgtagggagggactggtgcacttaaggttgggggaagagtgctgagccagagctgggacccgtggctgaagtgcccatttccctgtgaccaggccaggatctgtggtggtacaattgactctggccttccgagaaggtaccatcaatgtccacgacgtggagacacagttcaatcagtataaaacggaagcagcctctcgatataacctgacgatctcaagacgtcagcggtgaggctacttccctgctgcagccagcaccatgccggggcccctctccttccagtgtctgggtccccgctctttccttagtgctggcagcgggaggggcgcctcctctgggagactgccctgaccactgcttttccttttagtgagtgatgtgccatttcctttctctgaccagtctggggctggggtgccaggctggggcatcgcgctgctggtgctggtctgtgttctggttgcgctggccattgtctatctcattgccttggtgagtgcagtccctggccctgatcagagccccccggtagaaggcactccatggcctgccataacctcctatctccccaggctgtctgtcagtgccgccgaaagaactacgggcagctggacatctttccagcccgggatacctaccatcctatgagcgagtaccccacctaccacacccatgggcgctatgtgcccctagcagtaccgatcgtagcccctatgagaaggtgagattgggccccacaggcaggggaagcagagggtttggctgggcaaggattctgaagggggtacttggaaaacccaaagagcttggaagaggtgagaagtggcgtgaagtgagcaggggagggctggcaaggatgaggggcagaggtcagaggagttttgggggacaggcctgggaggagactatggaagaaaggggcccctcaaaagggagtgccccactgccagaattc

SEQ ID NO: 2 - protein, 307 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAQGVTSAPETRPPPGSTAPPAHGVTSAPDNRPALASTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMVSIGLSFPMLP

SEQ ID NO: 3 - DNA, 3108 bp, Homo sapiens
gggccccccacccacacaagcacagcaccgattgctgagttgaccacatccaatcctccgcctgagtcctcaacccctcagacctctcggtccacctcttcccctctcacggagtcaaccacccttctgagtaccctaccacctgccattgagatgaccagcacggccccaccctccacacccacggcacccacgaccacgagcggaggccacacactgtctccaccgcccagcaccaccacgtcccctccaggcacccccactcgcggtaccacgaccgggtcatcttcagcccccacccccagcactgtgcagacgaccaccaccagtgcctggaccccaacgccgaccccactctccacacccagcatcatcaggaccacaggcctgaggccctacccttcctctgtgcttatctgctgtgtcctgaacgacacctactacgcaccaggtgaggaggtgtacaacggcacatacggagacacctgttatttcgtcaactgctcactgagctgtacgttggagttctataactggtcctgcccatccacgccctccccaacacccacgccctccaagtcgacgcccacgccttccaagccatcgtccacgccctccaagccgacgcccggcaccaagccccccgagtgcccagactttgatcctcccagacaggagaacgagacttggtggctgtgcgactgcttcatggccacgtgcaagtacaacaacacggtggagatcgtgaaggtggagtgtgagccgccgcccatgcccacctgctccaacggcctccaacccgtgcgcgtcgaggaccccgacggctgctgctggcactgggagtgcgactgctactgcacgggctggggcgacccgcactatgtcaccttcgacggactctactacagctaccagggcaactgcacctacgtgctggtggaggagatcagcccctccgtggacaacttcggagtttacatcgacaactaccactgcgatcccaacgacaaggtgtcctgtccccgcaccctcatcgtgcgccacgagacccaggaggtgctgatcaagaccgtgcatatgatgcccatgcaggtgcaggtgcaggtgaacaggcaggcggtggcactgccctacaagaagtacgggctggaggtgtaccagtctggcatcaactacgtggtggacatccccgagctgggtgtcctcgtctcctacaatggcctgtccttctccgtcaggctgccctaccaccggtttggcaacaacaccaagggccagtgtggcacctgcaccaacaccacctccgacgactgcattctgcccagcggggagatcgtctccaactgtgaggctgcggctgaccagtggctggtgaacgacccctccaagccacactgcccccacagcagctccacgaccaagcgcccggccgtcactgtgcccgggggcggtaaaacgaccccacacaaggactgcaccccatctcccctctgccagctcatcaaggacagcctgtttgcccagtgccacgcactggtgcccccgcagcactactacgatgcctgcgtgttcgacagctgcttcatgccgggctcgagcctggagtgcgccagtctgcaggcctacgcagccctctgtgcccagcagaacatctgcctcgactggcggaaccacacgcatggggcctgcttggtggagtgcccatctcacagggagtaccaggcctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaagcccgttacccactgcgtggaagacggcacctacctcgccacggaggtcaaccctgccgacacctgctgcaacattaccgtctgcaagtgcaacaccagcctgtgcaaagagaagccctccgtgtgcccgctgggattcgaagtgaagagcaagatggtgcctggaaggtgctgtcccttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaactgcacattcttcagctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttgctttccaataataaactcagggggacatgc

SEQ ID NO: 4 - protein, 984 aa, Homo sapiens
GPPTHTSTAPIAELTTSNPPPESSTPQTSRSTSSPLTESTTLLSTLPPAIEMTSTAPPSTPTAPTTTSGGHTLSPPPSTTTSPPGTPTRGTTTGSSSAPTPSTVQTTTTSAWTPTPTPLSTPSIIRTTGLRPYPSSVLICCVLNDTYYAPGEEVYNGTYGDTCYFVNCSLSCTLEFYNWSCPSTPSPTPTPSKSTPTPSKPSSTPSKPTPGTKPPECPDFDPPRQENETWWLCDCFMATCKYNNTVEIVKVECEPPPMPTCSNGLQPVRVEDPDGCCWHWECDCYCTGWGDPHYVTFDGLYYSYQGNCTYVLVEEISPSVDNFGVYIDNYHCDPNDKVSCPRTLIVRHETQEVLIKTVHMMPMQVQVQVNRQAVALPYKKYGLEVYQSGINYVVDIPELGVLVSYNGLSFSVRLPYHRFGNNTKGQCGTCTNTTSDDCILPSGEIVSNCEAAADQWLVNDPSKPHCPHSSSTTKRPAVTVPGGGKTTPHKDCTPSPLCQLIKDSLFAQCHALVPPQHYYDACVFDSCFMPGSSLECASLQAYAALCAQQNICLDWRNHTHGACLVECPSHREYQACGPAEEPTCKSSSSQQNNTVLVEGCFCPEGTMNYAPGFDVCVKTCGCVGPDNVPREFGEHFEFDCKNCVCLEGGSGIICQPKRCSQKPVTHCVEDGTYLATEVNPADTCCNITVCKCNTSLCKEKPSVCPLGFEVKSKMVPGRCCPFYWCESKGVCVHGNAEYQPGSPVYSSKCQDCVCTDKVDNNTLLNVIACTHVPCNTSCSPGFELMEAPGECCKKCEQTHCIIKRPDNQHVILKPGDFKSDPKNNCTFFSCVKIHNQLISSVSNITCPNFDASICIPGSITFMPNGCCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLPTGTSRRARRSPRHLGSG

SEQ ID NO: 5 - DNA, 1574 bp, Homo sapiens
atcgctacgcccacttggtggcctataaaggaagcgggcgaaccccggcagccctacacaacttggggcccctctcctctccagcccttctcctgtgtgcctgcctcctgccgccgccaccatgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctgaggactcagctaccccggccggccacccaggaggcagggaggcagccgccccatctgccccacagtctccggcctctccagcctcagccccctgcttcagtcccttccccatgcttccttgcctgatgacaataaagcttgttgactcagctatg

SEQ ID NO: 6 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

(19) United States
(12) Patent Application Publication
(10) Pub. No.: US 2011/0256539 A1
(43) Pub. Date: Oct. 20, 2011

(54) Methods for Detecting Tumor Origin Based on MUC1, MUC2, and CK-17 Expression Levels

(76) Inventors: Hai-Su Yang, Westfield, NJ (US); Bruce Horten, New York, NY (US); Thomas Scholl, Westborough, MA (US)

(21) Appl. No.: 13/087,908

(22) Filed: Apr. 15, 2011

Related U.S. Application Data
(60) Provisional application No. 61/324,618, filed on Apr. 15, 2010.

(51) Int. Cl. C12Q 1/68; G01N 33/574; G01N 33/53

(52) U.S. Cl. 435/6.11; 435/7.1; 435/7.23

(57) ABSTRACT

Methods and compositions for detecting tumor origin are disclosed. In certain embodiments, the origin is determined by detecting expression levels of pan-epithelial membrane mucin (MUC1), intestinal-type secretory mucin (MUC2), cytokeratin 17 (CK17), or a combination thereof. Also disclosed are methods for aiding in the determination of an appropriate course of treatment for a subject with a tumor, or for predicting a therapeutic outcome of a subject, based on the expression levels of MUC1, MUC2, CK17, or a combination thereof. Kits for use in each of these methods are also provided.

### PRIOR RELATED APPLICATION

This application claims priority to U.S. provisional application 61/324,618, filed Apr. 15, 2010, the entire contents of which are hereby incorporated by reference.

### BACKGROUND OF THE INVENTION

Elucidating the origin of tumors is extremely important for effective treatment. It is particularly important for the treatment of pancreatic cancer. As a highly aggressive, treatment-refractory disease, pancreatic cancer is the fourth leading cause of cancer death in the U.S. It is seldom detected early, usually metastasizes quickly, and only in the relatively late clinically advanced stages of disease does it present with abdominal pain, weight loss, or jaundice. However, early in the course of the disease, pancreatic carcinomas directly infiltrate peripancreatic and retroperitoneal fatty tissues, duodenal, gastric and colonic walls, and invade perineural sheaths, the peritoneum, and lymphatic channels, eventually metastasizing to regional lymph nodes, the liver, and lungs.

Unlike other malignancies, such as breast, colorectal, and lung cancers in which there have been encouraging improvements in survival due to advances in adjuvant chemotherapy and radiation therapy, pancreatic carcinoma has had only a limited treatment response. This reflects not only the complexity of pancreatic cancer, but also the difficulty in correctly diagnosing the disease and accurately identifying the origin of metastatic adenocarcinomas.

Therefore, there is a great need for improved methods and systems for facilitating the diagnosis of a pancreatobiliary carcinoma. Also needed are highly specific biomarkers that can be used as detection tools for differential diagnosis of tumors originating in the pancreatobiliary system. There is also a need for improved methods for determining a course of treatment and predicting therapeutic outcomes for patients with tumors originating in the pancreatobiliary system.

### SUMMARY OF THE INVENTION

The present invention provides highly specific biomarkers for accurately distinguishing pancreatobiliary carcinomas from other primary site carcinomas based on the expression patterns of pan-epithelial membrane mucin (MUC1), intestinal-type secretory mucin (MUC2), and cytokeratin 17 (CK17). Thus, the present invention provides methods and compositions that can significantly improve the diagnosis, prognosis, and treatment of patients with pancreatic carcinomas.

In one aspect, the present invention provides a method of detecting a tumor (e.g., carcinoma), comprising detecting, in a biological sample obtained from a subject, expression levels of MUC1, MUC2, and CK17, as compared to controls; and determining the origin of the tumor in the biological sample based on the detected expression levels. In certain embodiments, the tumor is determined to be of pancreatobiliary origin if the detected expression levels are MUC1(+), MUC2(−), and CK17(+). In other embodiments, the tumor is determined to be originated from the colorectal region if the detected expression levels are MUC1(−), MUC2(+), and CK17(−). In some embodiments, the tumor is determined to be originated from the lung and/or kidney if the detected expression levels are MUC1(+), MUC2(−), and CK17(−).

In some embodiments of these methods, expression levels are determined by antibody immunohistochemistry. In other embodiments, expression levels are determined by nucleic acid hybridization. The expression levels may be determined qualitatively in certain embodiments. In some embodiments, expression levels are determined quantitatively.

In some embodiments, a biological sample suitable for the present invention is obtained from pancreas, peripancreatic fatty tissues, retroperitoneal fatty tissues, duodenal walls, gastric walls, colonic walls, peritoneum, lymphatic channels, lymph nodes, liver, kidney, lungs, or a combination thereof.

In some embodiments, the methods comprise negative controls. In certain embodiments, suitable negative controls are indicative of expression levels of MUC1, MUC2, and CK17 in normal tissues of the patient. In some embodiments, suitable negative controls are indicative of expression levels of MUC1, MUC2, and CK17 in the corresponding tissues from one or more healthy individuals.

In some embodiments, the methods comprise positive controls. In certain embodiments, suitable positive controls are indicative of expression levels of MUC1, MUC2, and CK17 in pancreatic carcinoma. In some embodiments, suitable positive controls are indicative of expression levels of MUC1, MUC2, and CK17 in colorectal cancer. In some embodiments, suitable positive controls are indicative of expression levels of MUC1, MUC2, and CK17 in lung cancer, kidney cancer, or both lung and kidney cancer.

In some embodiments, the present methods further include a step of determining a treatment for the subject based on the detected expression levels of MUC1, MUC2, and CK17. In certain embodiments, the present methods further include a step of predicting a therapeutic outcome for the subject.

In some embodiments, an inventive method according to the present invention is used in conjunction with an additional diagnostic method. For example, in certain embodiments, the methods are performed in conjunction with a diagnostic method detecting expression levels of carbohydrate antigen 19-9 (CA19-9).

In one aspect, the present invention provides a method of aiding a physician in determining an appropriate treatment for a patient with adenocarcinoma. In some embodiments, such a method comprises detecting, in a biological sample obtained from a patient, expression levels of MUC1, MUC2, and CK17, as compared to the controls; determining the origin of the tumor based on the detected expression levels; and reporting the determination of the origin of the tumor to the physician.

In another aspect, the present invention provides a kit for detecting a tumor (e.g., carcinoma), comprising a set of reagents that specifically detects expression levels of MUC1, MUC2, and CK17. In some embodiments, the set of reagents comprises antibodies, including fragments thereof, that specifically bind MUC1, MUC2, or CK17. In other embodiments, the set of reagents comprises nucleic acid probes. In some embodiments, the set of reagents comprise a combination of antibodies, including fragments thereof, and nucleic acid probes.

In some embodiments, a kit of the invention further includes a control indicative of expression levels of MUC1, MUC2, and CK17 in a healthy individual. In some embodiments, a kit of the invention further includes one or more controls indicative of expression levels of MUC1, MUC2, and CK17 in a cancer of known origin. In some embodiments, an kit further includes instructions for using said kit for determining the presence, absence, and/or the origin of the cancer or carcinoma.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The present invention may be better understood by reference to the following non-limiting figures. The drawings are for illustration purposes only, not for limitation.

FIG. 1 shows the results of exemplary immunostaining of MUC1, MUC2, and CK17 in pancreatobiliary adenocarcinomas. Panel A shows staining with antibody that binds MUC1; Panel B shows results of staining with antibody that binds MUC2; and Panel C shows results of staining with antibody that binds CK17. Panels A and C display positive cytoplasmic staining for MUC1 and CK17, while Panel B shows that MUC2 staining is absent (×200 magnification). MUC1 appears in most tumor cells predominantly at 3+ intensity. CK17 shows variable reactivity with intensity ranging from 1 to 3+. (1=weak, 2=moderate, 3=strong). This figure demonstrates that tumor samples from pancreatobiliary adenocarcinomas exhibit a pattern of MUC1 (+), MUC2 (−), and CK17 (+) expression.

FIG. 2 shows the results of exemplary expression of MUC1, MUC2, and CK17 in adenocarcinomas originating in the colorectal region. Panel A shows staining with antibody that binds MUC1; Panel B shows results of staining with antibody that binds MUC2; and Panel C shows results of staining with antibody that binds CK17. Tumors originating in the colorectal region show an immunoreactivity pattern of MUC1 (−), MUC2 (+), and CK17 (−). Panel B shows that MUC2 expression is predominantly cytoplasmic (×200 magnification). Panels A and B display only weak background staining

FIG. 3 shows the results of exemplary expression of MUC1, MUC2, and CK17 in adenocarcinomas of non-pancreatobiliary origin (i.e., renal cell carcinoma). Panel A shows staining with antibody that binds MUC1; Panel B shows results of staining with antibody that binds MUC2; and Panel C shows results of staining with antibody that binds CK17. Tumors of renal cell carcinoma display a MUC1 (+), MUC2 (−), and CK17 (−) reactivity pattern. Only MUC1 cytoplasmic immunostaining is detectable (Panel A); no staining with MUC2 or CK17 is observed (Panels B and C, respectively).

FIG. 4 shows the amino acid and nucleotide sequences for MUC1, MUC2, and CK17 in some embodiments of the present methods and kits.

### DETAILED DESCRIPTION

### Definitions

In this application, the use of “or” means “and/or” unless stated otherwise. As used in this application, the term “comprise” and variations of the term, such as “comprising” and “comprises,” are not intended to exclude other additives, components, integers or steps.

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3rd edition, Cold Spring Harbor Laboratory Press, N.Y., 2001.

In order for the present invention to be more readily understood, certain terms are first defined. Additional definitions for the following terms and other terms are set forth throughout the specification.

As used herein, the term “antibody” refers to a polypeptide consisting of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD, and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. An antibody can be specific for a particular antigen. The antibody or its antigen can be either an analyte or a binding partner. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of ordinary skill in the art will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked VH::VL heterodimer which may be expressed from a nucleic acid including VH- and VL-encoding sequences either joined directly or joined by a peptide-encoding linker. (See, e.g., Huston et al. (1988) Proc. Nat. Acad. Sci. USA, 85:5879-5883, the entire contents of which are herein incorporated by reference.) A number of structures exist for converting the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

As used herein, the terms “about” and “approximately” are used as equivalents. Any numerals used in this application with or without “about” or “approximately” are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “biological sample” is used herein in its broadest sense. A biological sample may be obtained from a subject (e.g., a human) or from components (e.g., tissues) of a subject. The sample may be of any biological tissue or fluid with which biomarkers of the present invention may be assayed. Frequently, the sample will be a “clinical sample”, i.e., a sample derived from a patient. Such samples include, but are not limited to, bodily fluids which may or may not contain cells, e.g., blood, blood products, urine, cell, and tissue material such as a cheek smear, Pap smear, fine needle aspiration, sternum puncture, and any other biopsied material taken during standard medical and open surgical procedures; and archival samples with known diagnosis, treatment, and/or outcome history. Biological samples may also include sections of tissues such as frozen sections taken from histological purposes. The term biological sample also encompasses any material derived by processing the biological sample. Derived materials include, but are not limited to, cells (or their progeny) isolated from the sample, proteins, or nucleic acid molecules extracted from the sample. Processing of the biological sample may involve one or more of filtration, distillation, extraction, concentration, inactivation of interfering components, addition of reagents, and the like.

As used herein, the term “biomarker” refers to a substance used as an indicator of a biological state. Typically, a biomarker is a characteristic that is objectively measured and evaluated as an indicator of normal biological processes, pathogenic processes, or pharmacologic responses to a therapeutic intervention. In certain embodiments, the biomarkers are pan-epithelial membrane mucin (MUC1), intestinal-type secretory mucin (MUC2), and cytokeratin 17 (CK17). “MUC1” refers to a protein as disclosed, for example, in SEQ ID NO:2, GenBank accession number AAB59612, or encoded by the nucleic acid disclosed as SEQ ID NO:1, GenBank accession number M35093.1. “MUC2” refers to a protein as disclosed, for example, in SEQ ID NO:4 or encoded by the nucleic acid disclosed as SEQ ID NO:3, GenBank accession number M94132. “CK17” refers to a protein as disclosed, for example, in SEQ ID NO:6, or encoded by the nucleic acid disclosed as SEQ ID NO:5.

As used herein, the terms “complement,” “complementary,” and “complementarity,” refer to the pairing of nucleotide sequences according to Watson/Crick pairing rules. For example, a sequence 5′-GCGGTCCCA-3′ has the complementary sequence of 5′-TGGGACCGC-3′. A complement sequence can also be a sequence of RNA complementary to the DNA sequence. Certain bases not commonly found in natural nucleic acids may be included in the complementary nucleic acids including, but not limited to, inosine, 7-deazaguanine, Locked Nucleic Acids (LNA), and Peptide Nucleic Acids (PNA). Complementary need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparison. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

As used herein, the term “diagnosis” refers to a process aimed at determining if an individual is afflicted with a disease, condition, or ailment.

As used herein, the term “epitope” refers to a fragment or portion of a molecule or a molecule compound (e.g., a polypeptide or a protein complex)) that makes contact with a particular antibody or antibody like protein.

As used herein, the term “hybridize” or “hybridization” refers to a process where two complementary nucleic acid strands anneal to each other under appropriately stringent conditions. Oligonucleotides or probes suitable for hybridizations typically contain 10-100 nucleotides in length (e.g., 18-50, 12-70, 10-30, 10-24, or 18-36 nucleotides in length). Nucleic acid hybridization techniques are well known in the art. See, e.g., Sambrook et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

The terms “labeled” and “labeled with a detectable agent or moiety” are used herein interchangeably to specify that an entity (e.g., a nucleic acid probe, antibody, etc.) can be visualized, for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, colorimetric labels, magnetic labels, haptens, molecular beacons, aptamer beacons, and the like.

As used herein, the term “multiplex PCR” refers to amplification of two or more regions which are each primed using a distinct primers pair.

The terms “nucleic acid,” “nucleic acid molecule,” and “polynucleotide” are used herein interchangeably. They refer to a deoxyribonucleotide or ribonucleotide polymer in either single-stranded or double-stranded form, and unless otherwise stated, encompass known analogs of natural nucleotides that can function in a similar manner as naturally occurring nucleotides. The terms encompass nucleic acid-like structures with synthetic backbones, as well as amplification products.

The term “probe,” as used herein, refers to a nucleic acid molecule of known sequence, which can be a short DNA sequence (i.e., an oligonucleotide), a PCR product, or mRNA isolate. Probes are specific DNA sequences to which nucleic acid fragments from a test sample are hybridized. Probes specifically bind to nucleic acids of complementary or substantially complementary sequence through one or more types of chemical bonds, usually through hydrogen bond formation.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

As used herein, the term “specific,” when used in connection with an oligonucleotide probe or primer, refers to an oligonucleotide or primer, under appropriate hybridization or washing conditions, is capable of hybridizing to the target of interest and not substantially hybridizing to nucleic acids which are not of interest. Higher levels of sequence identity are preferred and include at least 60%, 65%, 70%, 75%, 80%, 85%, 90%, 95%, 98%, 99%, or 100% sequence identity. In some embodiments, a specific oligonucleotide or primer contains at least 4, 6, 8, 10, 12, 14, 16, 18, 20, 22, 24, 26, 28, 30, 35, 40, 45, 50, 55, 60, 65, 70, or more bases of sequence identity with a portion of the nucleic acid to be hybridized or amplified when the oligonucleotide and the nucleic acid are aligned.

As used herein, the term “subject” refers to a human or any non-human animal. A subject can be a “patient,” which refers to a human presenting to a medical provider for diagnosis or treatment of a disease. A human includes pre and post natal forms. Particularly preferred subjects are humans being tested for the existence of a CFTR carrier state, CF disease or CF related disorder state.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “substantially complementary” refers to two sequences that can hybridize under stringent hybridization conditions. The skilled artisan will understand that substantially complementary sequences need not hybridize along their entire length. In some embodiments, “stringent hybridization conditions” refer to hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and SXDenhart's solution at 42° C. overnight; washing with 2×SSC, 0.1% SDS at 45° C.; and washing with 0.2×SSC, 0.1% SDS at 45° C. In some embodiments, stringent hybridization conditions should not allow for hybridization of two nucleic acids which differ over a stretch of 20 contiguous nucleotides by more than two bases.

An individual who is “suffering from” a disease, disorder, and/or condition has been diagnosed with or displays one or more symptoms of the disease, disorder, and/or condition.

An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Methods and Compositions

The present invention provides, among other things, methods and compositions for determining origins of tumors (e.g., carcinomas). The present invention encompasses the recognition that certain cancer types are difficult to distinguish from each other, which makes it difficult to choose proper treatment. In particular, prior to the present invention, the diagnosis, prognosis, and treatment of metastatic pancreatic carcinoma were daunting challenges. The signs of early stage pancreatic carcinoma are vague and often attributed to other problems by both patients and physicians. More specific symptoms tend to develop after the tumor has grown to invade other organs. Patients are usually diagnosed at an advanced stage, with a high incidence of associated metastatic spread throughout the body. As described in the Examples section, the present inventors discovered that the expression pattern of a biomarker panel including MUC1, MUC2, and CK17 can be used to accurately distinguish pancreatic cancer from cancers originated from other tissues such as colon, lung, and kidney. As described in the Examples, the inventors discovered that an expression pattern of MUC1 (+), MUC2 (−) and CK17 (+) can accurately indicate tumors originating in the pancreatobiliary system. In contrast, an expression pattern of MUC1 (−), MUC2 (+) and CK17 (−) correlates with tumors that arise from the colorectal region, while those originating from the lung and kidney have a MUC1 (+), MUC2 (−) and CK17 (−) expression profile. More surprisingly, using the expression pattern of MUC1 (+), MUC2 (−) and CK17 (+) resulted in greater sensitivity and specificity than CA 19-9, which is the only Food and Drug Administration (FDA)-approved marker for monitoring pancreatic carcinoma. Thus, the present invention represents a significant improvement in the field of pancreatic cancer diagnosis and treatment.

Various aspects of the invention are described in detail in the following sections. The use of sections is not meant to limit the invention. Each section can apply to any aspect of the invention. In this application, the use of “or” means “and/or” unless stated otherwise.

### MUC1

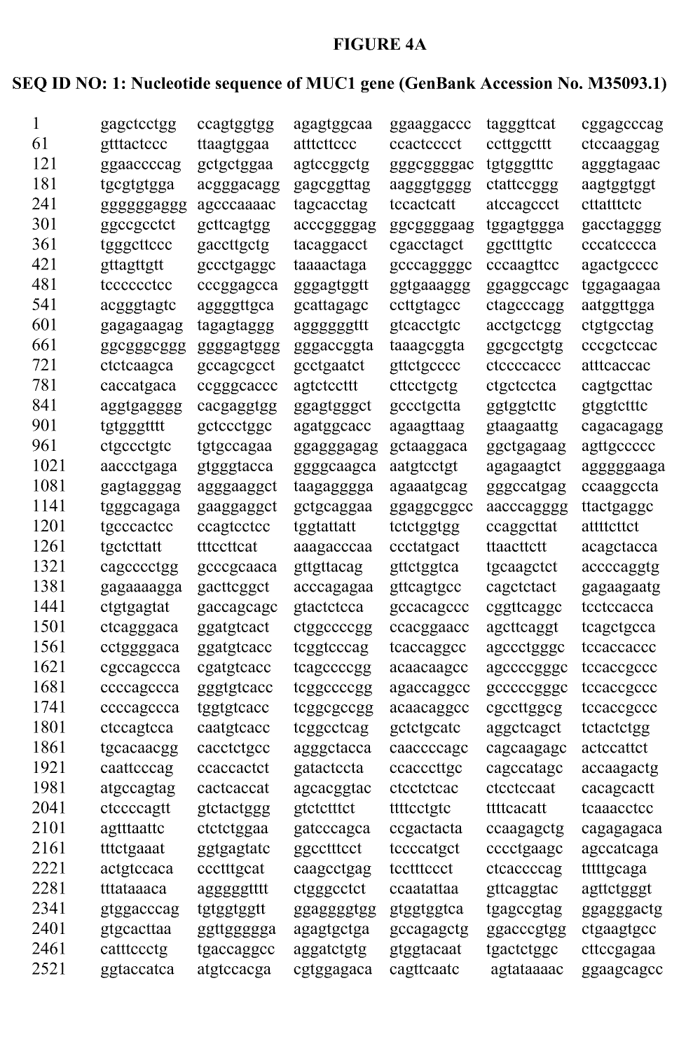

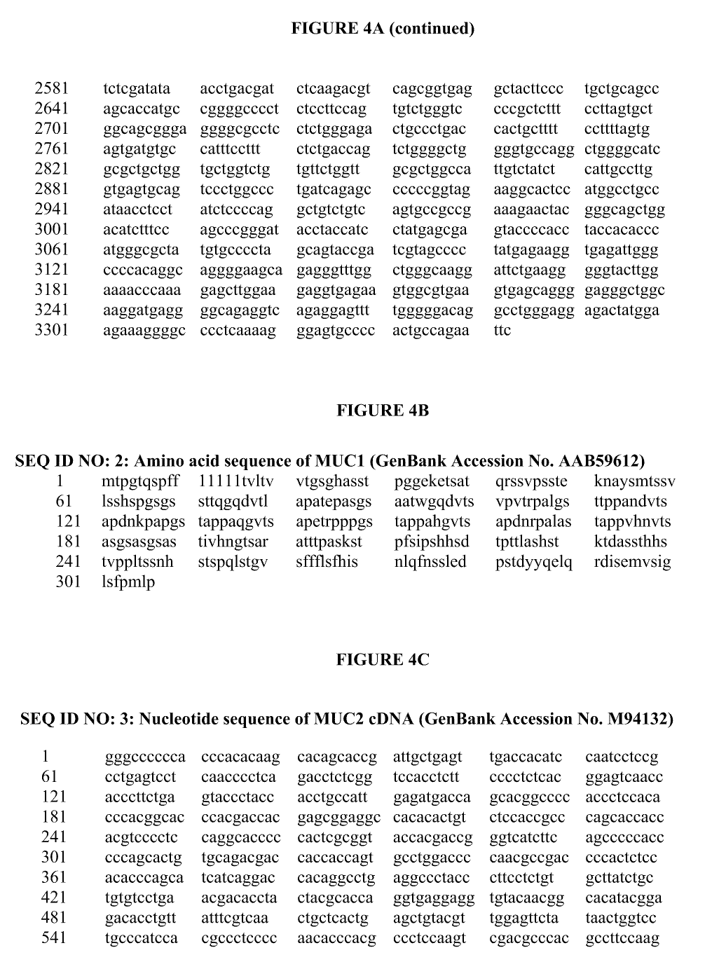

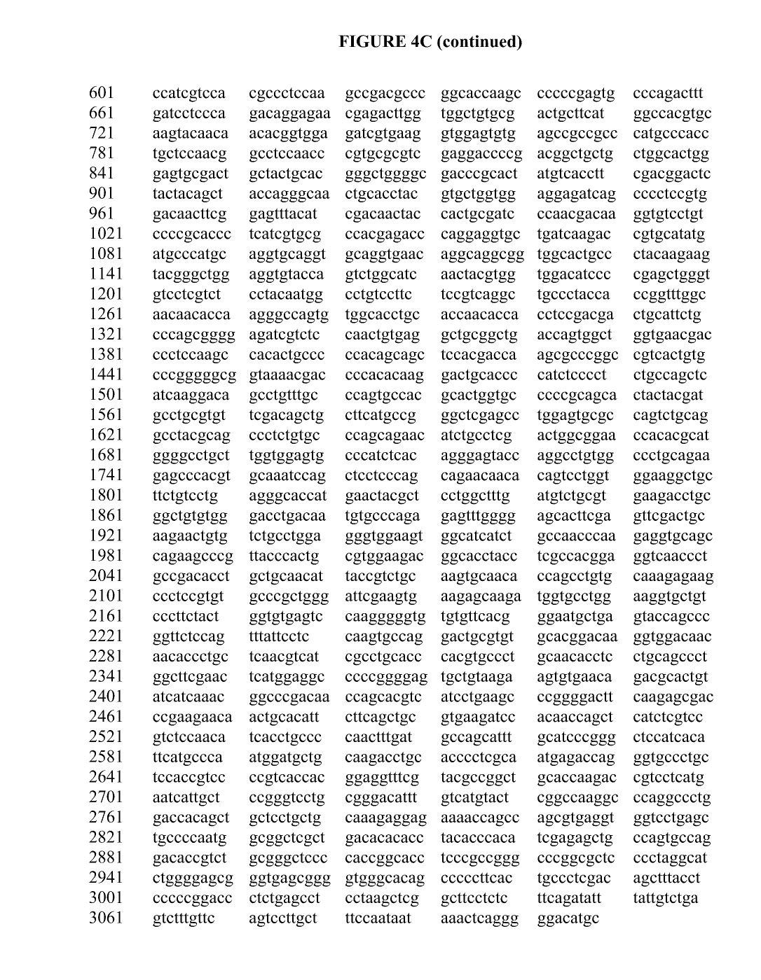

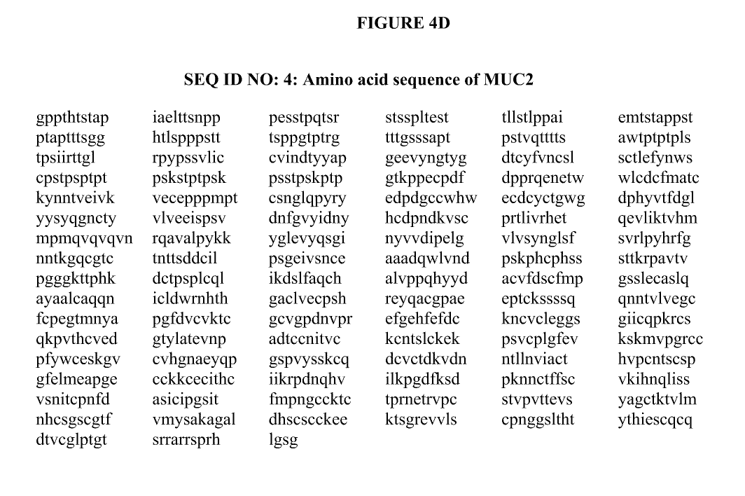

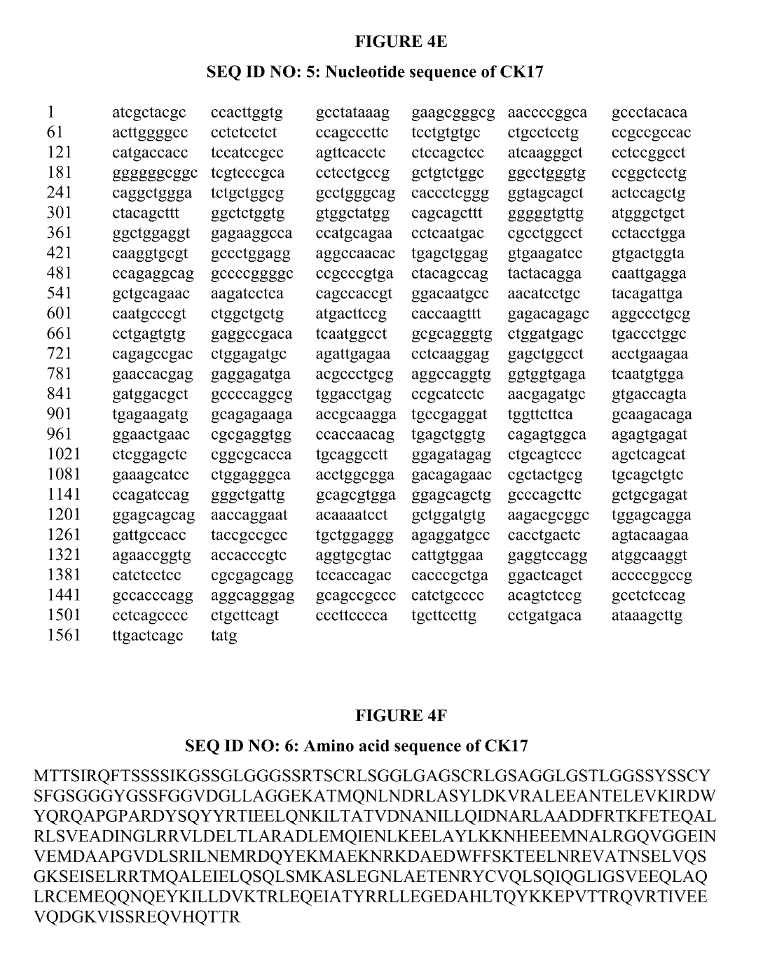

MUC1 is a type I transmembrane protein that includes a large extracellular subunit comprised of a mucin-type tandem repeat and a smaller subunit, which includes an extracellular domain, a transmembrane domain, and a cytoplasmic tail (Hollingsworth Mass. and Swanson B J. Mucins in cancer: protection and control of the cell surface. Nat Rev Cancer. 2004; 4:45-60.). In certain embodiments, the amino acid and nucleotide sequences of MUC1 are shown in FIG. 4A and FIG. 4B. In certain embodiments, the amino acid sequence or nucleotide sequence of MUC1 includes sequences that are at least 80%, 85%, 90%, 95%, 96%, 97%, 98%, and 99% identical to SEQ ID NO:1 or SEQ ID NO:2.

### MUC2

MUC2 is a gel-forming secretory mucin that is expressed in many organs, including the colon, small intestine and respiratory tract. See, Higashi M. et al. “Expression of MUC1 and MUC2 mucin antigens in intrahepatic bile duct tumors: its relationship with a new morphological classification of cholangiocarcinoma,” Hepatology. 1999; 30:1347-1355; and Hollingsworth Mass. and Swanson B J. “Mucins in cancer: protection and control of the cell surface,” Nat Rev Cancer. 2004; 4:45-60. In certain embodiments, the amino acid and nucleotide sequences of MUC2 are shown in FIG. 4C and FIG. 4D. In certain embodiments, the amino acid sequence or nucleotide sequence of MUC2 includes sequences that are at least 80%, 85%, 90%, 95%, 96%, 97%, 98%, and 99% identical to SEQ ID NO:3 or SEQ ID NO:4.

### CK17

Cytokeratins (CKs) are typical epithelial cell markers expressed in a tissue-specific and differentiation-dependent manner. CK17 is mainly expressed in squamous, basal, and transitional cell carcinomas, as well as in adenocarcinomas with squamous differentiation. See, Goldstein N. S. et al. “Cytokeratins 7, 17, and 20 reactivity in pancreatic and ampulla of vater adenocarcinomas. Percentage of positivity and distribution is affected by the cut-point threshold,” Am J Clin Pathol. 2001; 115:695-702. In certain embodiments, the amino acid and nucleotide sequences of CK17 are shown in FIG. 4E and FIG. 4F. In certain embodiments, the amino acid sequence or nucleotide sequence of MUC1 includes sequences that are at least 80%, 85%, 90%, 95%, 96%, 97%, 98%, and 99% identical to SEQ ID NO:5 or SEQ ID NO:6.

### Biological Samples and Detection of Biomarker Expression

Methods of the present invention may be applied to any type of biological samples including, but not limited to, urine, blood, blood products, feces and fecal material and extracts, saliva, cerebrospinal fluid, amniotic fluid, mucus, and cell and tissue material such as a cheek smear, Pap smear, fine needle aspiration, sternum puncture, and any other biopsied material taken during standard medical and open surgical procedures. In some embodiments, a suitable biological sample is a tumor or carcinoma sample obtained from pancreas, peripancreatic fatty tissues, retroperitoneal fatty tissues, duodenal walls, gastric walls, colonic walls, peritoneum, lymphatic channels, lymph nodes, liver, kidney, and/or lungs. Biological samples may be fresh or frozen samples collected from a subject, or archival samples with known diagnosis, treatment and/or outcome history. Biological samples may be collected by any non-invasive means, such as, for example, by drawing blood from a subject, or using fine needle aspiration or needle biopsy. Alternatively, biological samples may be collected by an invasive method, including, for example, surgical biopsy.

In certain embodiments, a crude biological sample without or with limited processing can be used for the present invention. In other embodiments, inventive methods according to the present invention can be performed at the single cell level (e.g., isolation of cells from the biological sample). Multiple biological samples may be taken from the same tissue or body part in order to obtain a representative sampling of the tissue.

In some embodiments, inventive methods are performed on a protein extract prepared from the biological sample. A suitable protein extract can be an extract containing the total protein content or fractionated protein extract containing one or more of membrane proteins, nuclear proteins, and cytosolic proteins. Methods of protein extraction are well known in the art (see, for example “Protein Methods”, D. M. Bollag et al., 2^{nd} Ed., 1996, Wiley-Liss; “Protein Purification Methods: A Practical Approach”, E. L. Harris and S. Angal (Eds.), 1989; “Protein Purification Techniques: A Practical Approach”, S. Roe, 2^{nd} Ed., 2001, Oxford University Press; “Principles and Reactions of Protein Extraction, Purification, and Characterization”, H. Ahmed, 2005, CRC Press: Boca Raton, Fla.). In some embodiments, after a protein extract has been obtained, the protein concentration of the extract is standardized to a value being the same as that of the control sample in order to allow signals of the protein markers to be compared and/or quantitated. Such standardization can be made using photometric or spectrometric methods or gel electrophoresis.

In some embodiments, inventive methods are performed on nucleic acids extracted from a biological sample. Typically, nucleic acids extracts suitable for the present invention contain RNA. For example, RNA may be extracted from the sample before analysis. Methods of RNA extraction are well known in the art (see, for example, J. Sambrook et al., “Molecular Cloning: A Laboratory Manual”, 1989, 2^{nd} Ed., Cold Spring Harbor Laboratory Press: Cold Spring Harbor, N.Y.). Most methods of RNA isolation from bodily fluids or tissues are based on the disruption of the tissue in the presence of protein denaturants to quickly and effectively inactivate RNases. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations.

In certain embodiments, after extraction, mRNA is amplified, and transcribed into cDNA, which can then serve as template for multiple rounds of transcription by appropriate RNA polymerases. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNA polymerases (such as avian myeloblastosis virus reverse transcriptase or Moloney murine leukemia virus reverse transcriptase).

### Determination of Protein Expression

In some embodiments, the present invention involve the determination of protein expression of MUC1, MUC2, and/or CK17 in a biological sample obtained from a subject. Determination of protein expression levels in the practice of the inventive methods may be performed by any suitable method (see, for example, E. Harlow and A. Lane, “Antibodies: A Laboratories Manual”, 1988, Cold Spring Harbor Laboratory: Cold Spring Harbor, N.Y.).

In general, protein expression levels are determined by contacting a biological sample isolated from a subject with binding agents for one or more of the protein markers; detecting, in the sample, the levels of polypeptides that bind to the binding agents; and comparing the levels of polypeptides in the sample with the levels of polypeptides in a control sample. As used herein, the term “binding agent” refers to an entity such as a polypeptide or antibody that specifically binds to an inventive protein marker. An entity “specifically binds” to a polypeptide if it reacts/interacts at a detectable level with the polypeptide but does not react/interact detectably with peptides containing unrelated sequences or sequences of different polypeptides.

In certain embodiments, the binding agent is a ribosome, with or without a peptide component, an RNA molecule, or a polypeptide (e.g., a polypeptide that comprises a polypeptide sequence of a protein marker, a peptide variant thereof, or a non-peptide mimetic of such a sequence).

In some embodiments, the binding agent is an antibody specific for a protein marker suitable for the invention (e.g., an antibody specific for MUC1, MUC2, or CK17). It is important that an antibody specific for MUC1 does not cross-react with MUC2 and an antibody specific for MUC2 does not cross-react with MUC1. Suitable antibodies for use in the methods of the present invention include monoclonal and polyclonal antibodies, immunologically active fragments (e.g., Fab or (Fab)₂ fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czernik et al., Neuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S, Neuberger et al., Nature, 1984, 312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity-purified by passage over a column to which a protein marker or fragment thereof is bound. The bound antibodies can then be eluted from the column using a buffer with a high salt concentration.

In certain embodiments, the binding agent is directly or indirectly labeled with a detectable moiety. The role of a detectable agent is to facilitate the detection step of the diagnostic method by allowing visualization of the complex formed by binding of the binding agent to the protein marker (or analog or fragment thereof). Preferably, the detectable agent is selected such that it generates a signal which can be measured and whose intensity is related (preferably proportional) to the amount of protein marker present in the sample being analyzed. Methods for labeling biological molecules such as polypeptides and antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available. Additional examples of detectable moieties or agents are provided in the Detectable Moieties section.

In certain embodiments, the binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

Protein expression levels in the diagnostic methods of the present invention may be determined using immunoassays. Examples of such assays include, but are not limited to, immunohistochemistry, time resolved fluorescence immunoassays (TR-FIA), radioimmunoassays, enzyme immunoassays (e.g., ELISA), immunofluorescence immunoprecipitation, latex agglutination, hemagglutination, which are conventional methods well-known in the art. As will be appreciated by one skilled in the art, an immunoassay may be competitive or non-competitive. Methods of detection and quantification of the signal generated by the complex formed by binding of the binding agent with the protein marker will depend on the nature of the assay and of the detectable moiety (e.g., fluorescent moiety).

In some embodiments, immunohistochemistry (IHC) is used to practice the methods of the invention. As used herein, immunohistochemistry refers to the process of localizing antigens (e.g., proteins) in cells of a tissue section based on the principle of specific antibody and antigen interactions. Typically, immunohistochemistry is performed on tissue slices. Depending on the purpose and the thickness of the sample, either thin (about 4-40 gm) slices are used, or if the tissue is not very thick and is penetrable, it may be used whole. Tissue slicing is usually accomplished through the use of a microtome, and slices are mounted on slides. In some embodiments, slices are used without mount. Such procedure is also known as “free-floating IHC.” Visualising an antibody-antigen interaction can be accomplished in a number of ways. In some emboidments, an antibody is conjugated to an enzyme, such as peroxidase, that can catalyse a colour-producing reaction (see immunoperoxidase staining section) In some embodiments, the antibody can also be tagged to a fluorophore, such as fluorescein, rhodamine, DyLight Fluor, or Alexa Fluor (see Detectable Moiety section).

In some embodiments, the protein expression levels may be determined using mass spectrometry based methods or image (including use of labeled ligand) based methods known in the art for the detection of proteins. Other suitable methods include proteomics-based methods. Proteomics, which studies the global changes of protein expression in a sample, can include the following steps: (1) separation of individual proteins in a sample by electrophoresis (1-D PAGE), (2) identification of individual proteins recovered from the gel (e.g., by mass spectrometry or N-terminal sequencing), and (3) analysis of the data using bioinformatics.

### Determination of Gene Expression

In some embodiments, inventive methods of the present invention may involve determination of the expression of MUC1, MUC2 or CK17 at gene expression levels. Determination of gene expression levels in the practice of the present invention may be performed by any suitable method. For example, determination of gene expression levels may be performed by detecting the expression of mRNA expressed from the genes of interest and/or by detecting the expression of a polypeptide encoded by the genes.

Various methods for detecting nucleic acid molecules can be used in the present invention including, but not limited to, Southern analysis, Northern analysis, polymerase chain reaction (PCR) (see, for example, U.S. Pat. Nos., 4,683,195; 4,683,202, and 6,040,166; “PCR Protocols: A Guide to Methods and Applications”, Innis et al. (Eds.), 1990, Academic Press: New York), reverse transcriptase PCR(RT-PCT), anchored PCR, competitive PCR (see, for example, U.S. Pat. No. 5,747,251), multiplex PCR, rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of polynucleotide sequences in biological samples may be constructed using conventional methods known in the art. Suitable probes may be based on nucleic acid sequences encoding a protein marker (e.g., MUC1, MUC2, or CK17) described herein or known in the art. Nucleic acid probes may comprise ribonucleic acids and/or deoxyribonucleic acids. In some embodiments, suitable nucleic acid probes are oligonucleotides (i.e., “oligonucleotide probes”). Generally, oligonucleotide probes are long enough to allow specific hybridization to target nucleic acids. Typically, the sizes of oligonucleotide probes vary from approximately 10 to 100 nucleotides. In some embodiments, oligonucleotide probes vary from 15 to 90, 15 to 80, 15 to 70, 15 to 60, 15 to 50, 15 to 40, 15 to 35, 15 to 30, 18 to 30, or 18 to 26 nucleotides in length. For example, in certain embodiments of the methods and kits of the invention, the probes comprise a fragment of 10-100, or 15 to 90, or 15 to 80, or 15 to 70, or 15 to 60, or 15 to 50, or 15 to 40, or 15 to 35, or 15 to 30, or 18 to 30, or 18 to 26 contiguous nucleotides in length of SEQ ID NOs:1, 3, or 5. Alternatively, a plurality of probes, each having sizes within these ranges, but derived from different regions of SEQ ID NO:1, SEQ ID NO:3, or SEQ ID NO:5 may be used in the methods and/or kits of the invention. As appreciated by those of ordinary skill in the art, the optimal length of an oligonucleotide probe may depend on the particular methods and/or conditions in which the oligonucleotide probe may be employed.

A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1:81-91; and S. Joos et al., J. Biotechnol. 1994, 35: 135-153).

Nucleic acid probes may be used in hybridization techniques to detect polynucleotides encoding the protein markers. The technique generally involves contacting and incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of nucleic acid molecules comprising polynucleotide sequences coding for a protein marker may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to the target sequence.

Alternatively, oligonucleotides or longer fragments derived from nucleic acids encoding each protein marker may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Hybridization and amplification techniques described herein may be used to assay qualitative and quantitative aspects of expression of nucleic acid molecules comprising polynucleotide sequences coding for the inventive protein markers.

### Detectable Moieties

In certain embodiments, certain molecules (e.g., nucleic acid probes, antibodies, etc.) used in accordance with and/or provided by the invention comprise one or more detectable entities or moieties, i.e., such molecules are “labeled” with such entities or moieties.

Any of a wide variety of detectable agents can be used in the practice of the present invention. Suitable detectable agents include, but are not limited to: various ligands, radionuclides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

In some embodiments, the detectable moiety is biotin. Biotin can be bound to avidins (such as streptavidin), which are typically conjugated (directly or indirectly) to other moieties (e.g., fluorescent moieties) that are detectable themselves.

Below are described some non-limiting examples of other detectable moieties.

Fluorescent Dyes

In certain embodiments, a detectable moiety is a fluorescent dye. Numerous known fluorescent dyes of a wide variety of chemical structures and physical characteristics are suitable for use in the practice of the present invention. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED™, SPECTRUM GREEN™, cyanine dyes (e.g., CY-3™, CY-5™, CY-3.5™, CY5.5™, etc.), ALEXA FLUOR™ dyes (e.g., ALEXA FLUOR™ 350, ALEXA FLUOR™ 488, ALEXA FLUOR™ 532, ALEXA FLUOR™ 546, ALEXA FLUOR™ 568, ALEXA FLUOR™ 594, ALEXA FLUOR™ 633, ALEXA FLUOR™ 660, ALEXA FLUOR™ 680, etc.), BODIPY™ dyes (e.g., BODIPY™ FL, BODIPY™ R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™ 630/650, BODIPY™ 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92: 4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described previously, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluoroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99 mTc, 111In, 1251, 1231, 1291, 1311, 1351, 186Re, 187Re, 201T1, 212Bi, 213Bi, 211At, 153Sm, 177Lu).

In some embodiments, signal amplification is achieved using labeled dendrimers as the detectable moiety (see, e.g., Physiol Genomics 3:93-99, 2000), the entire contents of which are herein incorporated by reference in their entirety. Fluorescently labeled dendrimers are available from Genisphere (Montvale, N.J.). These may be chemically conjugated to the oligonucleotide primers by methods known in the art.

### Clinical Utilities of Biomarker Panel MUC1, MUC2, and CK17

According to the present invention, expression patterns of a biomarker panel including MUC2, and/or CK17 can be used for diagnosis, prognosis and treatment of various carcinoma, in particular, pancreatic carcinoma.

In order to determine expression levels or patterns of biomarkers of interest (e.g., MUC1, MUC2, or CK17) for clinical uses, appropriate controls are typically used in various methods described herein. As used herein, the term “control” has its art-understood meaning of being a standard against which results are compared. Controls are generally used to augment integrity in experiments by isolating variables in order to make a conclusion about such variables. In some embodiments, a control is a reaction or assay that is performed simultaneously with a patient sample to be analyzed to provide a comparator. In some embodiments, a control is a historical one (i.e., of a test or assay performed previously, or an amount or result that is previously known). For example a control may be a mathematical numeral indicative of baseline expression level of a biomarker of interest. A control can also be a printed or otherwise saved record.

A control may be a positive control or a negative control. As used herein, a “negative control” refers to a control that indicates the baseline expression level (e.g., a protein or gene expression level) of a biomarker of interest (e.g., MUC1, MUC2, or CK17) under a normal or undiseased condition. For example, a suitable negative control can be the expression level of the biomarker of interest (e.g., MUC1, MUC2, or CK17) in normal tissues obtained from the same patient. In some embodiments, a suitable negative control can be the expression level of the biomarker of interest (e.g., MUC1, MUC2, or CK17) in the corresponding tissue obtained from a healthy individual. In other embodiments, a suitable negative control can be the expression level of a reference biomarker. Accordingly, the expression level of one or more reference biomarkers from the patient are compared to the expression level of the biomarker of interest (e.g., MUC1, MUC2, and CK17) to determine the relative expression levels of the biomarker. A suitable reference biomarker is one that exhibits similar expression levels under both normal and diseased conditions. The relative expression levels in the patient are then compared to relative expression levels of the same biomarkers in the patient's normal tissue(s) or from one or more healthy individual. As used herein, a “positive control” refers to a control that indicates the expression level (e.g., at protein or gene expression level) of a biomarker of interest (e.g., MUC1, MUC2, or CK17) in a tumor (e.g., carcinoma) tissue of known origin. For example, a suitable positive control is indicative of an expression level of a biomarker of interest (e.g., MUC1, MUC2, or CK17) in known pancreatic carcinoma. In some embodiments, a suitable positive control is indicative of an expression level of a biomarker of interest (e.g., MUC1, MUC2, or CK17) in colorectal cancer. In some embodiments, a suitable positive control is indicative of an expression level of a biomarker of interest (e.g., MUC1, MUC2, or CK17) in lung and/or kidney cancer.

Once the protein or gene expression level of a biomarker of interest is determined using methods described herein for the biological sample being analyzed, it can be compared to one or more controls. Comparison of expression levels according to methods of the present invention is preferably performed after the expression levels obtained have been corrected for both differences in the amount of sample assayed and variability in the quality of the sample used (e.g., amount of protein extracted, or amount and quality of mRNA tested). Correction may be carried out using different methods well-known in the art. For example, the protein concentration of a sample may be standardized using photometric or spectrometric methods or gel electrophoresis (as already mentioned above) before the sample is analyzed. In case of samples containing nucleic acid molecules, correction may be carried out by normalizing the levels against reference genes (e.g., housekeeping genes) in the same sample. Alternatively or additionally, normalization can be based on the mean or median signal (e.g., cycle threshold (Ct) in the case of RT-PCR) of all assayed genes or a large subset thereof (global normalization approach).

The expression level for a biomarker of interest (e.g., MUC1, MUC2, or CK17) in a patient sample can be determined qualitatively or quantititatively. In some embodiments, a digital scoring system can be used to record the intensity of various signals. For example, the expression level of a biomarker of interest can be qualitatively judged as positive (+) or negative (−) as compared to one or more controls by one or more laboratory personnel experienced in evaluating such results. In certain embodiments, signal intensity (such as staining, fluorescence, or the intensity of a band on a gel) indicative of expression levels of a biomarker of interest can be compared to appropriate controls to determine if the signal is negative or positive. Typically, a signal is considered positive if the signal is greater than that of a negative control. In some cases, positive signals can be further classified as weak, moderate or strong. An exemplary scoring system is shown in Table 1 below.

TABLE 1
Scoring System for Signal Intensity.

| Signal Intensity | Score |
|---|---|
| − (negative) | 0 |
| + (weak positive) | 1 |
| ++ (moderate) | 2 |
| +++ (strong) | 3 |

In some embodiments, the expression level of a biomarker of interest can be measured quantitatively. Depending on the methods used for measuring expression levels, means for collecting the signals and converting the signals to quantities of nucleic acids and proteins are well known in the art and can be adapted to practice the present invention. For example, guidance for making fluorescent intensity measurements and for relating them to quantities of analytes is available in the literature relating to chemical and molecular analysis, e.g. Guilbault, editor, Practical Fluorescence, Second Edition (Marcel Dekker, New York, 1990); Pesce et al., editors, Fluorescence Spectroscopy (Marcel Dekker, New York, 1971); White et al., Fluorescence Analysis: A Practical Approach (Marcel Dekker, New York, 1970); and the like.

Using the methods described herein, an expression pattern of a biomarker panel including MUC1, MUC2, and/or CK17 can be determined for a given patient sample, as compared to appropriate controls. Such expression patterns can be used to detect if the patient sample has cancer and/or correlate the cancer with appropriate tissue origins. Such a biomarker panel is particularly useful for distinguishing pancreatic cancer from other cancer, such as, for example, colon cancer, lung cancer, and kidney cancer. Exemplary expression patterns and their correlation with certain cancer type are shown in Table 2.

TABLE 2
Exemplary Expression Patterns

| Observed marker expression | | | |
|---|---|---|---|
| MUC1 | MUC2 | CK17 | Determined tumor origin |
| + | − | + | pancreatobiliary system |
| − | + | − | colorectal |
| + | − | − | Lung and/or kidney |

For example, the inventive biomarker panel can be used to distinguish metastasized pancreatic cancer from other localized primary cancer such as colon cancer, lung cancer, and kidney cancer. Additionally or alternatively, the inventive biomarker panel can also be used to distinguish early stage primary pancreatobiliary adenocarcinoma from cancer that has metastasized to the pancreas from a different tissue, such as colon, lung, or kidney.

Skilled physicians may select and prescribe treatments adapted to each individual patient based on the diagnosis and/or prognosis provided to the patient through determination of the expression patterns of a biomarker panel described herein. In particular, since methods disclosed herein allow for discrimination between early stage localized pancreatic cancer and metastasized cancers, these methods provide physicians with a non-subjective means to determine which patients may benefit from a more aggressive treatment, and which patients may be spared unnecessary interventions.

Selection of an appropriate therapeutic regimen for a given patient may be made based solely on the diagnosis or prognosis provided by the inventive methods described herein. Alternatively, appropriate therapeutic regimens may also be determined taking into consideration of consider other clinical or pathological parameters and/or biochemical factors used in existing methods to assess the risk of disease progression, including tumor stage, tumor grade, and the serum level of other markers such as CA19-9.

### Kits

The present invention provides kits comprising reagents useful for carrying out the methods of the invention. The diagnosis and prognosis methods described herein may be performed, for example, by diagnostic laboratories, experimental laboratories, or practitioners. The invention provides kits which can be used in these different settings.

Generally, kits of the invention comprise one or more reagents that specifically detect expression levels of MUC1, MUC2, and/or CK17. Suitable reagents may include, but are not limited to, antibodies or fragments thereof, and nucleic acid probes.

In some embodiments, provided kits further comprise reagents for carried out various detection methods described herein (e.g., immunohistochemistry, other immuno assays such as ELIZA, hybridization, PCR, etc.). For example, kits according to the invention may optionally contain buffers, enzymes, and/or reagents for use in methods described herein. For example, depending on the procedure, the kit may further comprise one or more of: extraction buffer and/or reagents, amplification buffer and/or reagents, hybridization buffer and/or reagents, reagents for preparing sample tissues for immunohistorchemistry analysis, immunodetection buffer and/or reagents, labeling buffer and/or reagents, and detection means. Protocols for using these buffers and reagents for performing different steps of the procedure may also be included in the kit.

Reagents may be supplied in a solid (e.g., lyophilized) or liquid form. Kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include samples of total protein, mRNA or tissue slides prepared from tissue of various physiological states, such as, for example, various normal tissue, benign tumor tissue, localized tumor tissue, and metastatic cancer tissue, to be used as controls. In some embodiments, a kit of the invention further includes a control indicative of expression levels of MUC1, MUC2, and/or CK17 in a healthy individual. In some embodiments, a kit of the invention further includes one or more controls indicative of expression levels of MUC1, MUC2 and/or CK17 in cancers of known origins (e.g., colorectal cancer, lung and/or kidney cancer). In some embodiments, an kit further includes instructions for how to determine the presence, absence and/or the origin of the cancer.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

### EXAMPLES

The following examples serve to illustrate the present invention. These examples are in no way intended to limit the scope of the invention.

### Example 1

### Immunohistochemical Procedures

Tissue samples were fixed in 10% neutral buffered formalin (NBF) and embedded in paraffin. Immunohistochemical procedures were performed using the automated BondMax system (Leica Microsystems Inc., Bannockburn, Ill.). Briefly, deparaffinization using BondMax was followed by an antigen retrieval step for 15 minutes using a high pH epitope retrieval buffer from Leica Microsystems Inc. (Bannockburn, Ill.). A polymer-based detection system used was the L-M Intense Kit with 3,3′-diaminobenzidine (DAB) as the chromogen (Leica Microsystems Inc., Bannockburn, Ill.). Slides were counterstained with hematoxylin. Appropriate positive and negative tissue control samples were used throughout the study. Mouse monoclonal antibodies, anti-MUC1 (1:250), anti-MUC2 (1:150), and anti-CK17 (1:200) were obtained from LeicaMicrosystems Novocastra Laboratories (Newcastle, UK). Mouse monoclonal antibody anti-CA19.9 (1:16000) was obtained from Covance (Princeton, N.J.).

In some cases, the immunoreactivity of MUC1, MUC2, or CK17 was evaluated based on the staining intensity of positive cells distributed in the tissues. Intensity of staining was scored as 0 for no staining (negative), and as 1, 2, or 3 for weak (+), moderate (++), or strong staining (+++), respectively.

### Example 2

### Expression of MUC1, MUC2, and CK17 in Normal Tissues

Previous studies have reported that MUC1, MUC1, and CK17 are expressed in various tissue types (Goldstein N. S. et al. Am J Gin Patna 2001; 115:695-702; Higashi M. et al. Hepatology. 1999; 30:1347-1355; Hollingsworth M. A. et al. Nat Rev Cancer. 2004; 4:45-60). In the present Example, the expression patterns of these proteins were evaluated in multiple tissues by immunohistochemical analysis using the method described in Example 1.

MUC1 immunoreactivity was primarily cytoplasmic in most of the tissues examined including the following: the basal and para-basal cell layer of squamous epithelium, the stratified esophageal layer (which exhibited weak staining), the luminal borders of gastric glands in the stomach, the dendritic network of Peyer's Patches in the small intestine, luminal borders of hepatic bile ducts, distal renal tubules, alveolar lining cells and mesothelial cells in lung ducts and mammary lobules, the squamous epithelium of the cervix, prostatic glands, the dendritic network of white pulp germinal centers in the spleen, eccrine sweat glands and sebaceous glands in the skin, and the lumina of ducts and acinar cells in salivary glands. In normal adult pancreas, MUC1 expression was confined to the apical membrane of ducts in luminal borders. MUC1 was also detected in cell types such as follicular cells of the thyroid, and the squamous epithelium and dendritic reticulum of germinal centers in tonsils.

CK17 displayed a strong cytoplasmic reactivity in the following tissues: the basal cell layer of the squamous epithelium in the esophagus, the squamous epithelium and basal layer of the bronchi in the lung, the myoepithelial cell layer of the breast ducts and lobules, the basal layer of the squamous epithelium and endocervical glands in the uterine cervix, the luminal borders of endometrial glands in the uterus, the basal cell layer of normal prostate glands, the squamous epithelium (Hassall's corpuscles) in the thymus, the basal layer of sweat glands in the skin, and the myoepithelial layer of the ducts and acini of salivary glands. All other epithelial, mesenchymal, and lymphoid tissues evaluated in this Example were not reactive to the CK17 antibody.

In contrast, MUC2 was expressed in epithelial surface and crypts of the small intestine and colon. No other epithelial, mesenchymal, or lymphoid organs evaluated in this particular Example showed MUC2 immunoreactivity.

Exemplary expression patterns of MUC1, MUC2, and CK17 are summarized in Table 3.

TABLE 3
Expression patterns of MUC1, MUC2^{a} and CK17 in normal tissues.

| Tissue | MUC1 | CK17^{b} |
|---|---|---|
| Breast | ducts and lobules | myoepithelial cell layer of breast |
| | | ducts and lobules |
| Cervix | squamous epithelium | basal layer of squamous epithelium |
| | | and endocervical glands |
| Esophagus | basal and para-basal cell layer of | focal in basal cell layer of squamous |
| | squamous epithelium; weak | epithelium |
| | reactivity of stratified layer | |
| Kidney | distal tubules | Negative |
| Liver | luminal borders of bile ducts | Negative |
| Lung | alveolar lining cells and | squamous epithelium and basal layer |
| | mesothelial cells | of bronchi |
| Pancreas | ducts in luminal borders | Negative |
| Prostate | prostatic glands | basal cell layer of normal prostate |
| | | glands |
| Salivary gland | luminal borders of ducts and | myoepithelial layer of ducts and |
| | acinar cells | acinar |
| Skin | eccrine sweat glands and | basal layer of sweat glands |
| | sebaceous glands | |
| Small intestine | dendritic network of Peyer's | Negative |
| | patches | |
| Spleen | dendritic network of white pulp and | Negative |
| | germinal centers | |
| Stomach | Luminal borders of gastric | Negative |
| | glands | |
| Thymus | Negative | squamous epithelium (Hassall's |
| | | corpuscles) |
| Thyroid | follicular cells | Negative |
| Tonsil | squamous epithelium and dendritic | Negative |
| | reticulum of germinal centers | |
| Uterus | Negative | luminal borders of endometrial |
| | | glands |

^{a}MUC2 was expressed in epithelial surface and crypts of the small intestine and colon. No other epithelial, mesenchymal or lymphoid organs evaluated in this particular Example showed MUC2 immunoreactivity.
^{b}All other epithelial, mesenchymal or lymphoid tissues were unreactive.

### Example 3

### Expression of MUC1, MUC2, and CK17 in Adenocarcinomas

A primary reason pancreatic adenocarcinoma is among the deadliest malignancies is the lack of symptoms for early detection and diagnosis. In addition, early local invasion and metastatic spread of the tumor make both surgery and other conventional treatment strategies inadequate for most patients. Thus, the identification of a tumor's primary site of origin is critical for appropriate treatment of this disease.

Adenocarcinomas from a variety of organs were collected and de-identified prior to their use. Tissue samples were fixed and stained as described above for Example 1. The immunoreactivity of MUC1, MUC2, or CK17 was evaluated based on the staining intensity of positive cells distributed in the carcinoma tissues. Intensity of staining was scored as 0 for no staining (negative), and as 1, 2, or 3 for weak (+), moderate (++), or strong staining (+++), respectively.

A MUC1, MUC2, and CK17 antigen expression panel was used to determine the primary site of origin for a series of metastatic adenocarcinomas obtained from multiple body sites such as the liver, lung, kidney, colon, and pancreas.

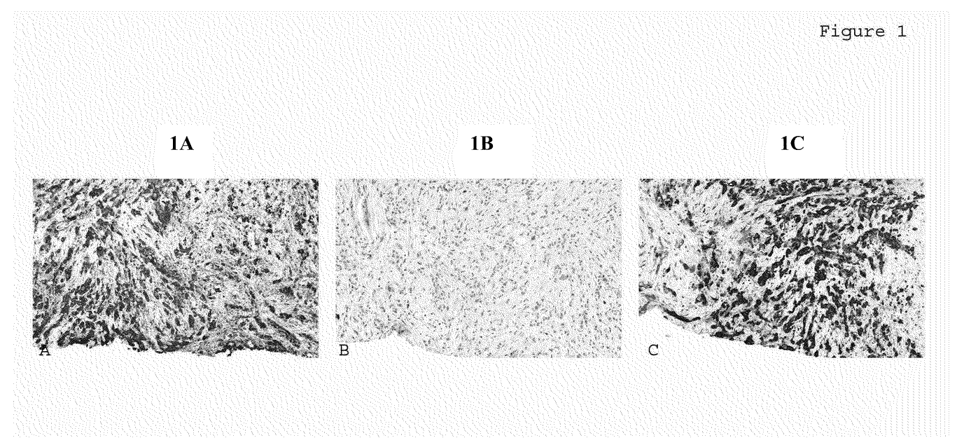

Immunoreactivity for all three markers was seen in the cytoplasm. Tumors originating in the pancreatobiliary system resulted in a pattern of MUC1 (+), MUC2 (−), and CK17 (+). MUC1 was seen in most of the tumor cells predominantly at 3+intensity. MUC2 expression was negative in all tissues and CK17 showed variable reactivity ranging from 1-3+in intensity (FIG. 1).

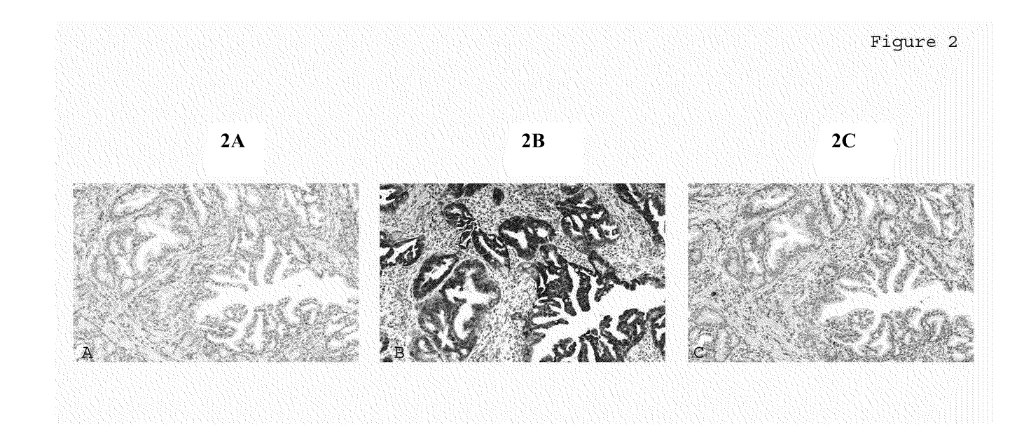

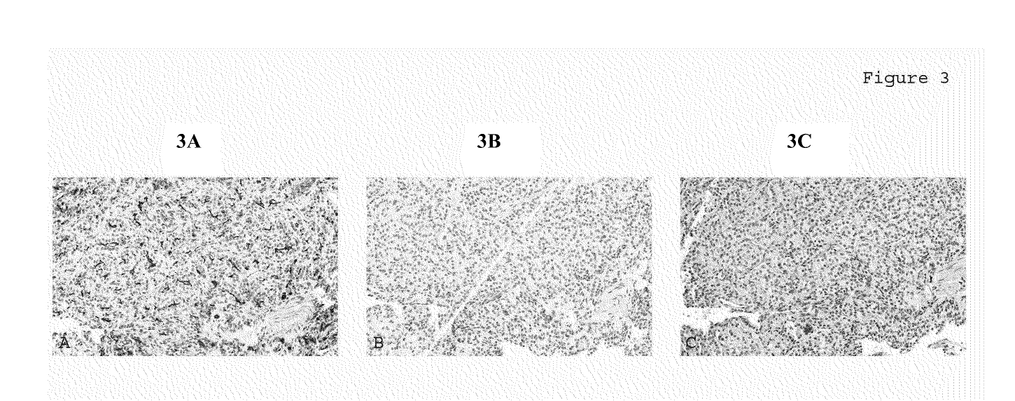

Tumors arising from colorectal carcinoma showed an immunoreactivity pattern consisting of MUC1 (−), MUC2 (+), and CK17 (−). MUC2 expression was predominantly cytoplasmic. None of these neoplasms displayed any reactivity with MUC1 and CK17 (FIG. 2). Moreover, tumors of non-pancreatobiliary origin including those from the lung (data not shown) and kidney exhibited an expression profile consisting of MUC1 (+), MUC2 (−), and CK17 (−) (FIG. 3).

In order to evaluate any processing variability in MUC1, MUC2, and CK17 staining, a comparative study was performed in two laboratories with a subset of the adenocarcinoma specimens. A consistent immunoreactive staining pattern was obtained in both laboratories for MUC1, MUC2 and CK17. MUC1 predominately stained the non-mucinous epithelium of the pancreas, lung, pancreatic adenocarcinoma, renal cells (clear cell type) and mesothelium. Mucinous adenocarcinomas displayed focal reactivity. MUC2 showed positive staining in mucinous colonic adenocarcinomas and negative staining in all other tumors Immunoreactivity of CK17 was mainly localized in non-mucinous adenocarcinomas, the basal epithelium of the bronchus, squamous cells, and mesothelium (data not shown).

### Discussion

These data show that in the normal adult pancreas, MUC1 expression is concentrated in the apical membrane of intralobular ductules, possibly as a manifestation of the “lumen maintenance” role of MUC1 (Table 1). In addition, MUC1 was highly expressed in the cytoplasm in a series of pancreatic adenocarcinoma cases (FIG. 1, Table 2). Previous studies have revealed that MUC1 is overexpressed during the early stages of pancreatic carcinoma development and is further increased in invasive carcinoma (Levi E. et al. J Clin Pathol. 2004; 57:456-462). Moreover, it is abundant in almost all examples of conventional ductal adenocarcinoma (Levi E. et al. J Clin Pathol. 2004; 57:456-462).

The pattern of mucin expression within the pancreas varies depending on the pathophysiological condition. It is noteworthy that MUC2 expression was not detected in the normal pancreas in our studies. Among the tissue samples studied in this Example, MUC2 cytoplasmic staining was only observed on epithelial surfaces and in the crypts of the small intestine and colon (Table 1). In addition, these data indicated that MUC2 displayed positive immunoreactivity in tumors originating only in the colorectal region, and not the pancreatobiliary system (FIG. 2).

CK17 was positively expressed in the basal cell layer and myoepithelial layer in multiple organs including the esophagus, lung, breast, cervix, uterus, prostate, thymus, skin, and salivary glands (Table 1). Moreover, CK17 immunostaining was also detected in adenocarcinomas originating from the pancreatobiliary system (FIG. 1).

Elucidating the origin of adenocarcinomas is important for effective treatment. However, cases with mixed histological patterns are frequently encountered. Immunostaining can thus be valuable in identifying the origin of a carcinoma, particularly when tumor histomorphology is not discriminatory. MUC1, MUC2, and CK17 cytoplasmic staining are not usually seen in normal or preinvasive neoplasia or other carcinomas in this region. The data shown in this example indicate that analysis of the individual staining for these three markers can be combined and used as a diagnostic tool to distinguish tumors originating in the pancreatobiliary system from those of other sites. These results further reveal that the expressions of MUC1 and CK17 are very specific for pancreatobiliary tumors, and that MUC2 is a sensitive marker for tumors of intestinal origin.

### Example 4

### MUC1, MUC2, and CK17 Expression Panel is More Sensitive than CA19-9

A current immunohistochemistry (IHC) marker for diagnosis of pancreatic related tumors is CA 19-9. CA 19-9 is predominantly used despite its low specificity for these tumors since it is also elevated in gastrointestinal, ovary, lung, and bladder carcinomas (Safi F et al. Hepatogastroenterology. 1998; 45:253-259). The correlation of CA19-9 expression was compared with that of the MUC1, MUC2, and CK17 panel in a series of pancreatic adenocarcinomas by immunohistochemistry analysis. Table 4 summarizes the characteristics of the study population and their expression patterns. A total of 51 patients, ranging from 41 to 86 years, who had been diagnosed with pancreatic adenocarcinomas were tested. The incidence for disease and age was highly correlated, underscoring the aggressive nature of the disease.

TABLE 4
Clinical Utility of MUC1, MUC2, and CK17 expression as
markers for pancreatic adenocarcinomas in 51 patients.

| | MUC1(+) | | MUC1(+) | |
|---|---|---|---|---|
| | MUC2(−) | | MUC2(−) | |
| | CK17 (+) | | CK17 (−) | |
| Patient | CA19-9(+) | CA19-9(−) | CA19-9(+) | Total |
| Age (yrs) | | | | |
| <55 | 4 (8%) | 2 (4%) | 0 (0%) | 6 (11%) |
| 55-65 | 11 (22%) | 1 (2%) | 0 (0%) | 12 (24%) |
| >65 | 19 (37%) | 10 (19%) | 4 (8%) | 33 (65%) |
| Gender | | | | |
| Male | 11 (22%) | 5 (9%) | 2 (4%) | 18 (35%) |
| Female | 23 (45%) | 8 (16%) | 2 (4%) | 33 (65%) |

Sixty-seven percent of the tumors originating in the pancreatobiliary system were identified by the immunostaining pattern of MUC1 (+), MUC2 (−) and CK17 (+) and also correlated with positive CA19-9 staining. An additional 25% of those tumors that were MUC1 (+), MUC2 (−) and CK17 (+) displayed no immunoreactivity for CA19-9. This result clearly demonstrates that the MUC1 (+), MUC2 (−), and CK17 (+) panel appears to be a more sensitive immunohistochemical marker for adenocarcinomas with pancreatobiliary origin than CA19-9 positivity.

### Discussion

Early detection or diagnosis is critical for the treatment of adenocarcinomas, with the highest cure rate reported for those tumors that are truly localized to the pancreas. However, this stage of disease accounts for fewer than 20% of cases due, in part, to the lack of highly specific tumor markers for the detection of pancreatic adenocarcinoma. Indeed, CA19-9 is the only Food and Drug Administration (FDA)-approved marker for monitoring pancreatic carcinoma, yet its elevated levels in other cancers have made early diagnosis challenging.

Previous studies have found that the CA19-9 epitope occurs within the MUC1 protein and that an anti-CA19-9 antibody recognizes a mucin-type glycoprotein (Burdick M D et al. J Biol. Chem. 1997; 272:24198-24202). The present data show that most of the adenocarcinomas that we identified as being of pancreatobiliary origin via the MUC1 (+), MUC2 (−) and CK17 (+) immunostaining pattern also displayed positive reactivity for CA 19-9. More significantly, an additional subset of the pancreatic adenocarcinomas cases that also exhibited MUC1 (+), MUC2 (−), and CK17 (+) expression did not display any immunoreactivity with CA19-9. These tumors are expected to also be of pancreatobiliary origin. These results support the use of the MUC1, MUC2, and CK17 panel as a valuable detection tool for the differential diagnosis of tumors originating in the pancreatobiliary system.

### Example 5

### Determination of Cancer Origin Using an Antibody Panel for MUC1, MUC2, and CK17

Examples 1-4 establish the utility of MUC1, MUC2, and CK17 expression patterns for determining tissue origin of cancers. In the present example, an antibody panel for MUC1, MUC2, and CK17 expression is used to determine the tissue origin of adenocarcinoma samples. Tissues are processed and stained for MUC1, MUC2, and CK17 as described in Example 1. Staining is then qualitatively judged as positive (+) or negative (−) by one or more laboratory personnel experienced in evaluating immunohistochemistry staining. Staining patterns are used to determine tissue origin using correlations as shown in Table 5. An appropriate course of treatment is determined based on the tissue origin of the adenocarcinoma.

TABLE 5
Staining pattern and corresponding tumor origin.

| Observed marker expression | | | |
|---|---|---|---|
| MUC1 | MUC2 | CK17 | Determined tumor origin |
| + | − | + | pancreatobiliary system |
| − | + | − | colorectal |
| + | − | − | lung and/or kidney |

### Example 6

### Determination of MUC1, MUC2, and CK17 Expression by RT-PCR

In the present Example, MUC1, MUC2, and CK17 messenger RNA (mRNA) expression is used to determine tissue origin of adenocarcinomas. Adenocarcinoma tissue is obtained from human patients and then either freshly processed to extract RNA, or frozen and then immediately processed upon thawing to extract RNA using standard molecular biology techniques known in the art. RNA may be further processed or purified (e.g., to select for RNA species that have a polyA tail, thus enriching for mRNA).

MUC1, MUC2, and CK17 mRNA is reverse-transcribed into cDNA using oligo-dT primers. Resulting cDNA is then quantitated using real-time PCR. Labeled nucleic acid probes specific for MUC1, MUC2, or CK17 transcripts are used to hybridize MUC1, MUC2, or CK17 during amplification. Probes are dual-labeled with a fluorescent moiety and a quencher that quenches signal from the fluorescent moiety. As amplification reactions proceed, probes bind to their targets during the annealing phase and then are subsequently degraded by exonuclease activity, which releases fluorescent moieties from the probes (and therefore the vicinity of the quencher). Fluorescent signals are detected in real-time as PCR is conducted, and give a semi-quantitative or quantitative read on the amount of MUC1, MUC2, and CK17 mRNA.

Expression of MUC1, MUC2, and CK17 is determined as positive or negative based on predetermined threshold values and/or based on comparison with one or more control samples. Expression patterns are then used to determine tissue origin using correlations shown in Table 5. An appropriate course of treatment is determined based on the tissue origin of the adenocarcinoma.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a,” “an,” and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

What is claimed is:

1. A method of detecting origin of a tumor, comprising:
(a) detecting, in a biological sample obtained from a subject, expression levels of pan-epithelial membrane mucin (MUC1), intestinal-type secretory mucin (MUC2), and cytokeratin 17 (CK17);
(b) comparing expression levels of MUC1, MUC2, and CK17 to a control; and
(c) determining the origin of the tumor based on the detected expression levels.

2. The method of claim 1, wherein the tumor is determined to be of pancreatobiliary origin if the detected expression levels are MUC1(+), MUC2(−), and CK17(+).

3. The method of claim 1, wherein the tumor is determined to be originated from the colorectal region if the detected expression levels are MUC1(−), MUC2(+), and CK17(−).

4. The method of claim 1, wherein the tumor is determined to be originated from the lung and/or kidney if the detected expression levels are MUC1(+), MUC2(−), and CK17(−).

5. The method of claim 1, wherein the expression levels are detected by at least one of the methods selected from the group consisting of antibody immunohistochemistry, nucleic acid hybridization, or tissue staining.

6. The method of claim 1, wherein the expression levels are determined qualitatively.

7. The method of claim 1, wherein the expression levels are determined quantitatively.

8. The method of claim 1, wherein the biological sample is from the pancreas, peripancreatic fatty tissues, retroperitoneal fatty tissues, duodenal walls, gastric walls, colonic walls, peritoneum, lymphatic channels, lymph nodes, liver, kidney, lungs, or a combination thereof.

9. The method of claim 1, wherein the control comprises a negative control.

10. The method of claim 9, wherein the negative control is indicative of expression levels of MUC1, MUC2, and CK17 in normal tissues of the subject or in the corresponding tissues of a healthy individual.

11. The method of claim 1, wherein the control comprises a positive control.

12. The method of claim 11, wherein the positive control is indicative of expression levels of MUC1, MUC2, and CK17 in pancreatic carcinoma.

13. The method of claim 11, wherein the positive control is indicative of expression levels of MUC1, MUC2, and CK17 in colorectal cancer.

14. The method of claim 11, wherein the positive control is indicative of expression levels of MUC1, MUC2, and CK17 in lung cancer, kidney cancer, or both lung and kidney cancer.

15. The method of claim 1, further comprising determining a course of treatment for the patient based on the detected expression levels.

16. The method of claim 1, further comprising predicting a therapeutic outcome for the patient based on the detected expression levels.

17. The method of claim 1 wherein the method is used in conjunction with an additional diagnostic method.

18. The method of claim 17, wherein the additional diagnostic method comprises detection of the expression level of carbohydrate antigen 19-9 (CA 19-9).

19. A method for aiding a physician in determining an appropriate treatment for a patient with adenocarcinoma comprising:
detecting, in a biological sample obtained from the patient, expression levels of pan-epithelial membrane mucin (MUC1), intestinal-type secretory mucin (MUC2), and cytokeratin 17 (CK17), as compared to controls;
determining the origin of the tumor based on the detected expression levels of MUC1, MUC2, and CK17; and
reporting the determination of the origin of the tumor to the physician.

20. A kit for detecting origin of a tumor, comprising:
a set of reagents that specifically detects expression levels of pan-epithelial membrane mucin (MUC1), intestinal-type secretory mucin (MUC2), and cytokeratin 17 (CK17).

21. The kit of claim 20, wherein the reagents comprise antibodies, nucleic acid probes, or a combination thereof.

22. The kit of claim 20, wherein the kit further comprises a control indicative of expression levels of MUC1, MUC2, and CK17 in a healthy individual; a control indicative of expression levels of MUC1, MUC2, and CK17 in a cancer of known origin; or a combination thereof.
